(19) United States
(12) Patent Application Publication
(10) Pub. No.: US 2015/0198616 A1
(43) Pub. Date: Jul. 16, 2015

(54) METHODS OF DIAGNOSING AND DETERMINING RISK OF DEVELOPING DISSEMINATED INTRAVASCULAR COAGULATION

(71) Applicant: THE GENERAL HOSPITAL CORPORATION, Boston, MA (US)

(72) Inventor: Kazue Takahashi, Cambridge, MA (US)

(21) Appl. No.: 14/323,793

(22) Filed: Jul. 3, 2014

Related U.S. Application Data
(60) Provisional application No. 61/925,680, filed on Jan. 10, 2014.

(51) Int. Cl. G01N 33/68

(52) U.S. Cl. CPC G01N 33/6893; G01N 2800/52; G01N 2333/47

(57) ABSTRACT

The present invention relates to assays, methods, and systems for non-invasive and early diagnosis of disseminated intravascular coagulation (DIC). By measuring the CL-K1 level in a sample obtained from a subject and comparing it with a reference level, one can identify whether a subject has DIC or a risk of developing DIC if the CL-K1 level is above the reference level. In some embodiments, the sample is a plasma sample. The present invention also relates to assays, methods, and systems for monitoring the CL-K1 levels in the subject.

### CROSS-REFERENCE TO RELATED APPLICATION

This application claims benefit under 35 U.S.C. §119(e) of U.S. Provisional Application No. 61/925,680 filed Jan. 10, 2014, the contents of which are incorporated herein by reference in its entirety.

### TECHNICAL FIELD

The present invention relates generally to diagnosis of disseminated intravascular coagulation (DIC) and assessing the risk of developing DIC.

### BACKGROUND

Disseminated intravascular coagulation (DIC) is a severe medical complication in which thrombosis (coagulation) and bleeding occur simultaneously. There is a strong association with multiple organ failure and a very high mortality rate of about 70% (Toh C H, et al., Intensive Care Med 2003, 29, 55-61; Smith E Y, et al., Am J Clin Pathol 2004, 12, 138-141; Penner J A, Semin Thromb Hemost 1998, 24, 45-52). However, there are multiple challenges facing DIC diagnosis. First, there are no reliable biomarkers or risk factors for DIC. Second, not all patients with similar clinical conditions develop DIC. Third, even though an early stage of DIC can be revealed by a sensitive test that identifies an abnormal biphasic waveform in activated partial thromboplastin time (aPTT) testing, it is available only on the MDA analyzer which is not routinely available to most laboratories. In an aPTT test, the waveform represents the change in light transmittance through a plasma specimen as the aPTT reaction takes place. The biphasic waveform is characterized by an initial steep negative slope, which is not seen in a normal waveform and is caused, in part, by precipitation of a C-reactive protein (CRP) complex (Toh C H, et al., Intensive Care Med 2003, 29, 55-61; Smith E Y, et al., Am J Clin Pathol 2004, 12, 138-141; Matsumoto T, et al., Clin Appl Thromb Hemost 2006, 12, 185-192). The abnormal biphasic waveform was predictive of approximately half of fully developed DIC 18 hours prior to the diagnosis provided by a battery of several other clinical tests that are currently regarded as definitive of DIC (Toh C H, et al., Blood 2002, 100, 2522-2529).

Accordingly, novel methods for diagnosing DIC or assessing the risk of developing DIC in a subject would be beneficial.

### SUMMARY

Embodiments of various aspects described herein are based, inter alia, on the identification of a biomarker called collectin kidney 1 (CL-K1), the level of which can be used for disseminated intravascular coagulation (DIC) diagnosis. More specifically, the inventors discovered that plasma CL-K1 levels are elevated in patients with DIC, as compared to the levels in patients without DIC. Accordingly, embodiments of various aspects described herein provide, among other things, assays, methods, and systems for determining whether a subject has DIC or a risk of developing DIC. These assays, methods, and systems described herein can be non-invasive and simple, as they can be performed on a body fluid sample collected from a subject (e.g., a blood sample or a plasma sample). Since early diagnosis is critical for patients suffering from DIC, the assays, methods, and systems described herein can permit physicians to diagnose DIC in patients early and provide an appropriate treatment in a timely manner.

One aspect provided herein relates to an assay comprising: measuring, in a sample obtained from a subject, a level of collectin kidney 1 (CL-K1); comparing the level of CL-K1 with a reference level; and identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.

In some embodiments where the level of CL-K1 is determined to be above the reference level, the assay can further comprise determining that the subject does not have a respiratory disease if the level of CL-K1 is above the reference level. In some embodiments where the level of CL-K1 is determined to be above the reference level, the assay can further comprise determining that the subject does not have coagulopathy other than DIC.

In some embodiments, the assay can further comprise providing a treatment appropriate for treating DIC when the subject is diagnosed with DIC or determined to have a risk for DIC. For example, in some embodiments, the treatment can comprise administering an anticoagulant. In some embodiments, the treatment can comprise administering recombinant factor VII. In some embodiments, the treatment can comprise transfusion of platelets or fresh frozen plasma.

In some embodiments, the assay can further comprise determining an underlying condition/cause of DIC in the subject.

In some embodiments, the sample is a blood sample or a plasma sample.

In some embodiments, the level of CL-K1 is measured by an immunoassay.

In some embodiments, the immunoassay is an enzyme-linked immunosorbent assay (ELISA).

In some embodiments, the ELISA is sandwich ELISA using two antibodies specific to CL-K1.

In some embodiments, the sandwich ELISA comprises: contacting the sample obtained from the subject with a first anti-CL-K1 antibody; washing the sample to remove excess unbound antigen; and contacting the sample with a second anti-CL-K1 antibody.

In some embodiments, the reference level corresponds to an average CL-K1 level in a population of healthy subjects.

In some embodiments, the reference level is two standard deviations above an average CL-K1 level in a population of healthy subjects.

In some embodiments, the reference level corresponds to an average CL-K1 level in a population of subjects without DIC.

In some embodiments, the subject without DIC also does not have a respiratory disease or coagulopathy.

In some embodiments, the reference level is two standard deviations above an average CL-K1 level in a population of subjects without DIC.

In some embodiments, the reference level is about 619 ng/mL.

In some embodiments, the reference level corresponds to a level of CL-K1 measured in the subject at an earlier time point.

In some embodiments, the subject is a mammal.

In some embodiments, the mammal is a human.

In some embodiments, the subject has a vascular disease.

Another aspect of the invention relates to an assay comprising: contacting a plasma sample obtained from a subject with a detectable anti-CL-K1-antibody reagent; detecting the intensity of a detectable signal, wherein the intensity of the detectable signal indicates a level of CL-K1 in the subject; comparing the level of CL-K1 with a reference level; and identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.

In some embodiments, the antibody reagent is detectably labeled or capable of generating a detectable signal.

In some embodiments, the assay further comprises administering, to the subject, a treatment selected from the group consisting of: an anticoagulant, recombinant factor VII, and transfusion of platelets or fresh frozen plasma.

Yet another aspect of the invention relates to a method of identifying whether a subject has disseminated intravascular coagulation (DIC) or a risk of developing DIC, the method comprising: assaying, in a sample obtained from a subject, a level of collectin kidney 1 (CL-K1); comparing the level of CL-K1 with a reference level; and identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.

In some embodiments, the method further comprises determining that the subject does not have a respiratory disease or coagulopathy other than DIC if the level of CL-K1 is above the reference level.

In some embodiments, the method further comprises providing a treatment appropriate for treating DIC.

In some embodiments, the method further comprises determining an underlying condition for DIC in the subject.

In some embodiments, the treatment comprises administering an anticoagulant.

In some embodiments, the treatment comprises administering recombinant factor VII.

In some embodiments, the treatment comprises transfusion of platelets or fresh frozen plasma.

In some embodiments, the sample is a blood sample or a plasma sample.

In some embodiments, the level of CL-K1 is measured by an immunoassay.

In some embodiments, the immunoassay is an enzyme-linked immunosorbent assay (ELISA).

In some embodiments, the ELISA is sandwich ELISA using two antibodies specific to CL-K1.

In some embodiments, the sandwich ELISA comprises: contacting the sample obtained from the subject with a first anti-CL-K1 antibody; washing the sample to remove excess unbound antigen; and contacting the sample with a second anti-CL-K1 antibody.

In some embodiments, the reference level corresponds to an average CL-K1 level in a population of healthy subjects.

In some embodiments, the reference level is two standard deviations above an average CL-K1 level in a population of healthy subjects.

In some embodiments, the reference level corresponds to an average CL-K1 level in a population of subjects without DIC.

In some embodiments, the subject without DIC also does not have a respiratory disease or coagulopathy.

In some embodiments, the reference level is two standard deviations above an average CL-K1 level in a population of subjects without DIC.

In some embodiments, the reference level is about 619 ng/mL.

In some embodiments, the reference level corresponds to a level of CL-K1 measured in the subject at an earlier time point.

In some embodiments, the subject is a mammal.

In some embodiments, the mammal is a human.

In some embodiments, the subject has a vascular disease.

In another aspect, the invention provides a method of monitoring treatment progress in a subject suffering from disseminated intravascular coagulation (DIC), the method comprising: measuring, at a first time point, a first level of collectin kidney 1 (CL-K1) in a first sample obtained from the subject; measuring, at a second time point, a second level of CL-K1 in a second sample obtained from the subject, wherein the second time point is later than the first time point and after the administration of a therapeutic agent for DIC, and wherein if the second level is significantly lower than the first level, then the treatment is considered to be effective.

In some embodiments, the first sample and the second sample are blood samples or plasma samples.

In some embodiments, the subject is a human.

In another aspect, a method is provided herein to monitor a risk of a subject of developing disseminated intravascular coagulation (DIC), the method comprising: measuring, at a first time point, a first level of collectin kidney 1 (CL-K1) in a first sample obtained from the subject; measuring, at a second time point, a second level of CL-K1 in a second sample obtained from the subject, wherein the second time point is later than the first time point, and wherein if the second level is significantly higher than the first level, then the risk of the subject developing DIC is identified as increased.

In some embodiments, the first sample and the second sample are blood samples or plasma samples.

In some embodiments, the subject is a human.

In yet another aspect, a computer system is provided herein for determining whether a subject has disseminated intravascular coagulation (DIC) or a risk of developing DIC, the system comprising: a determination module configured to measure a CL-K1 level in a test sample obtained from the subject; a storage module configured to store output data from the determination module; a comparison module adapted to compare the data stored on the storage module with a reference level, and to provide a retrieved content; and a display module for displaying the retrieved content.

In some embodiments, the determination module measures the intensity of a detectable signal from an immunoassay indicating the CL-K1 level.

In some embodiments, the reference level corresponds to an average CL-K1 level in a population of healthy subjects.

In some embodiments, the reference level is two standard deviations above an average CL-K1 level in a population of healthy subjects.

In some embodiments, the reference level corresponds to an average CL-K1 level in a population of subjects without DIC.

In some embodiments, the subject without DIC also does not have a respiratory disease or coagulopathy.

In some embodiments, the reference level is two standard deviations above the average CL-K1 level in a population of subjects without DIC.

In some embodiments, the reference level is about 619 ng/mL.

In some embodiments, the reference level corresponds to a level of CL-K1 measured in the subject at an earlier time point.

In some embodiments, if the CL-K1 level is above the reference level, the subject is identified as having a risk of developing DIC.

In some embodiments, if the CL-K1 level is above the reference level, the subject is identified as having DIC and in need of treatment for DIC.

### BRIEF DESCRIPTION OF THE DRAWINGS

FIG. 1A is a plot comparing the plasma CL-K1 levels in the DIC group with those in the non-DIC group. Numbers in parentheses indicate specimen numbers. Statistical significance was analyzed by a nonparametric Wilcoxon/Kruskal-Wallis test and only p values less than 0.05 are indicated.

FIG. 1B is a plot that divides the populations in FIG. 1A by male or female gender. Numbers in parentheses indicate specimen numbers. Statistical significance was analyzed by a nonparametric Wilcoxon/Kruskal-Wallis test and only p values less than 0.05 are indicated.

FIG. 2 is a plot comparing the plasma CL-K1 levels in a healthy control with those in the non-DIC group.

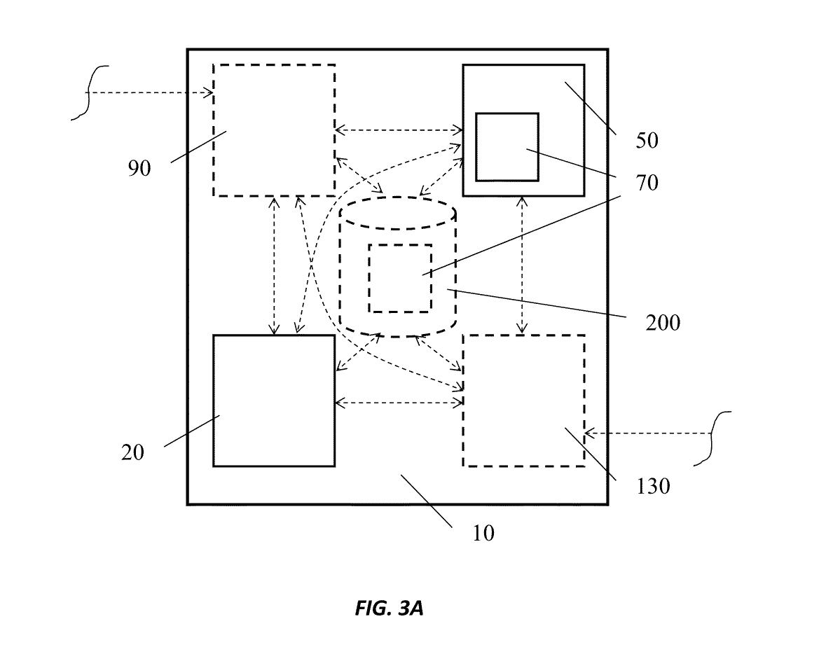

FIGS. 3A-3B are block diagrams showing an example of a system for determining a Cl-K1 level in a test sample of a subject as described herein.

FIG. 4 is a block diagram showing exemplary instructions on a computer readable medium for determining a Cl-K1 level in a test sample of a subject as described herein.

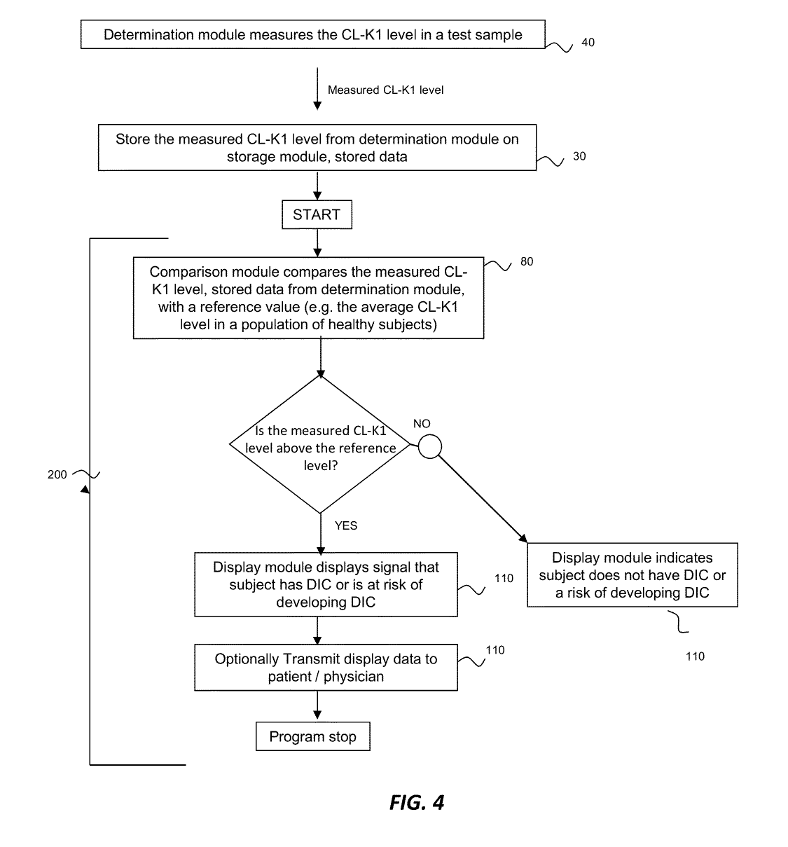

### DETAILED DESCRIPTION

The invention is based, inter alia, on the discovery that the level of collectin kidney 1 (CL-K1) is significantly elevated in patients with DIC. Accordingly, embodiments of the invention provide, among other things, assays and methods for determining whether a subject has DIC or a risk of developing DIC by measuring the level of collectin (e.g., CL-K1) in a sample obtained from the subject.

CL-K1, also known as collectin 11, is a recently identified collectin that is synthesized in most organs and circulates in blood. CL-K1 is an innate immune molecule that may play a significant role in host defense. There are at least nine types of collectins: MBL, SP-A, SP-D, CL-L1, CL-P1, CL-43, CL-46, CL-K1, and conglutinin.

As used herein, the term “CL-K1” generally refers to a CL-K1 polypeptide or a CL-K1 polynucleotide that is similar or identical to the sequence of a wild-type CL-K1.

In some embodiments, the term “CL-K1” refers to a CL-K1 polypeptide having an amino acid sequence that is at least 70% or more (including at least 75%, at least 80%, at least 85%, at least 90%, at least 95%, at least 97%, at least 99%, or 100%) identical to that of a wild-type CL-K1.

In some embodiments, the term “CL-K1” refers to a CL-K1 polynucleotide having a nucleotide sequence that is at least 70% or more (including at least 75%, at least 80%, at least 85%, at least 90%, at least 95%, at least 97%, at least 99%, or 100%) identical to that of a wild-type CL-K1 or a portion thereof, and encodes a CL-K1 polypeptide as described herein.

The wild-type CL-K1 sequences of various species are available on the world wide web from the NCBI, including human, and can be found, for example, in US20120015400.

Where the term “CL-K1” refers to a CL-K1 polypeptide, the term “CL-K1 polypeptide” also encompasses a portion or fragment of such a CL-K1 polypeptide that retains at least about 70% or more (including at least 75%, at least 80%, at least 85%, at least 90%, at least 95%, at least 97%, at least 99%, or 100%) of the functional activity of the wild-type CL-K1 polypeptide.

In various aspects described herein, methods for measuring CL-K1 or a fragment thereof from a sample are known in the art, including, but not limited to mRNA expression using PCR or real-time PCR, protein analysis using western blot, immunoassay, and/or ELISA, and/or sequencing analysis. Thus, in some embodiments, nucleic acid molecules can be isolated from a patient's sample to measure CL-K1 mRNA expression, or proteins can be isolated to measure CL-K1 protein expression.

In one aspect, provided herein is a method of identifying whether a subject has disseminated intravascular coagulation (DIC) or a risk of developing DIC, the method comprising: measuring, in a sample obtained from a subject, a level of collectin kidney 1 (CL-K1); comparing the level of CL-K1 with a reference level; and identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.

In some embodiments, if the level of CL-K1 is found to be above the reference level, the method further comprises determining that the subject does not have a respiratory disease or coagulopathy other than DIC. As used herein, the term “coagulopathy” is a disorder in which the ability of blood to clot is impaired. Coagulopathy generally includes blood clotting disorder and bleeding disorder including, but is not limited to, DIC, acquired platelet function defects, acquired platelet function defects, acquired platelet function defects, Factor II deficiency, Factor V deficiency, Factor VII deficiency, Factor X deficiency, Factor XII deficiency, hemophilia A, hemophilia B, idiopathic thrombocytopenic purpura (ITP), Von Willebrand's disease (types I, II, and III), thrombophilia (e.g., Factor V Leiden), protein C deficiency, protein S deficiency, anti-thrombin deficiency, and prothrombin 20210A mutations. In general, for DIC, thrombosis and bleeding occur uncontrollably, and can result in massive bleeding and thrombosis.

In some embodiments, if the level of CL-K1 is found to be above the reference level, the method can further comprise determining that the subject does not have a respiratory disease, acquired platelet function defects, acquired platelet function defects, acquired platelet function defects, Factor II deficiency, Factor V deficiency, Factor VII deficiency, Factor X deficiency, Factor XII deficiency, hemophilia A, hemophilia B, idiopathic thrombocytopenic purpura (ITP), Von Willebrand's disease (types I, II, and III), thrombophilia (e.g., Factor V Leiden), protein C deficiency, protein S deficiency, anti-thrombin deficiency, or prothrombin 20210A mutations.

CL-K1 levels can be measured using a variety of methods known in the art including, but not limited to, an immunological test, surface plasmon resonance, and photonic crystal-based detection. Immunological tests include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments, the immunoassay can be a quantitative or a semi-quantitative immunoassay.

An immunoassay is a biochemical test that measures the concentration of a substance in a biological sample, typically a fluid sample such as plasma, using the interaction of an antibody or antibodies to its antigen. The assay takes advantage of the highly specific binding of an antibody with its antigen. In some embodiments, specific binding of the CL-K1 molecule with an anti-CL-K1 antibody forms a CL-K1-antibody complex. The complex can then detected by a variety of methods known in the art. An immunoassay also often involves the use of a detection antibody. Examples of anti-CL-K1 antibodies can be found in US20120015400 and Yoshizaki et al., J. Biochem. 2012, 151, 57-64, and they can be used for the purposes of the methods and assays described herein to measure CL-K1 levels. Anti-CL-K1 antibodies are also commercially available through vendors such as OriGene, Novus Biologicals, Abcam, ThermoFisher, and LSBio. Examples of anti-collectin antibodies can also be found in U.S. Pat. No. 7,985,833.

In some embodiments, CL-K1 levels are measured by ELISA, also called enzyme immunoassay or EIA. ELISA is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (i.e. CL-K1) can be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a sandwich ELISA is used. By way of non-limiting example, a sandwich ELISA to determine the level of CL-K1 in a sample can be performed as follows: a well plate (e.g., 24, 96, or 384) is coated with a first anti-CL-K1 antibody. After rinsing and blocking of non-specific binding sites, a sample (e.g., a plasma sample) is added to the well plate and allowed to incubate for a period of time. Rinse the sample to remove any excess unbound antigen. A second antibody (e.g., biotinylated anti-CL-K1) is added to the well plate followed by the addition of ABC-AP (available at Vector Lab, Burlingame, Calif.). Finally p-nitrophenyl phosphate substrates are added to the well plate to produce para-nitrophenol, which is yellow and can be assayed for absorbance at 405 nm using a plate reader (e.g., M3 plate reader by Molecular Devices). The level of CL-K1 can then be calculated from the absorbance. For example, a sandwich ELISA method of measuring CL-K1 levels is reported by Yoshizaki et al. (J. Biochem. 2012, 151, 57-64) and US20120015400, the contents of each of which are incorporated herein by reference in its entirety.

There are other different forms of ELISA, which are well known to those skilled in the art. Standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference for their teachings on ELISA.

In one embodiment, the levels of CL-K1 in a sample can be detected by a lateral flow immunoassay test (LFIA), also known as the immunochromatographic assay, or strip test. LFIAs are a simple device intended to detect the presence (or absence) of antigen, e.g. CL-K1, in a fluid sample. There are currently many LFIA tests used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of CL-K1 present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

A typical test strip consists of the following components: (1) sample application area comprising an absorbent pad (i.e., the matrix or material) onto which the test sample is applied; (2) conjugate or reagent pad—this contains antibody reagent(s) specific to the target antigen (e.g. specific CL-K1 or fragment thereof) which can be conjugated to colored particles (e.g., colloidal gold particles, or latex microspheres); (3) test results area comprising a reaction membrane—typically a hydrophobic nitrocellulose or cellulose acetate membrane onto which antibody reagents (e.g., anti-CL-K1 antibodies) are immobilized in a line across the membrane as a capture zone or test line (a control zone may also be present, containing antibodies specific for the antibody reagents conjugated to the particles or microspheres); and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line which contains an antibody that picks up free latex/gold in order to confirm the test has operated correctly.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of CL-K1 using antibody reagents as described herein.

A urine dipstick is a colorimetric chemical assay that can be used to determine the pH, specific gravity, protein, glucose, ketone, bilirubin, urobilinogen, blood, leukocyte, and nitrite levels of an individual's urine. It consists of a reagent stick-pad, which is immersed in a fresh urine specimen and then withdrawn. After predetermined times the colors of the reagent pad are compared to standardized reference charts. The urine dipstick offers an inexpensive and fast method to perform screening urinalyses, which help in identifying the presence of various diseases or health problems. A urine dipstick provides a simple and clear diagnostic guideline and can be used in the methods and kits as described herein. Accordingly, one aspect of the present technology relates to a method for detecting CL-K1 using a device, such as a dipstick, as described herein.

Other techniques can be used to detect the level of CL-K1 in a sample. One such technique is the dot blot, and adaptation of Western blotting (Towbin et at., Proc. Nat. Acad. Sci. 76:4350 (1979)). In a Western blot, CL-K1 can be dissociated with detergents and heat, and separated on an SDS-PAGE gel before being transferred to a solid support, such as a nitrocellulose or PVDF membrane. The membrane is incubated with an antibody reagent specific for CL-K1. The membrane is then washed to remove unbound proteins and proteins with non-specific binding. Detectably labeled enzyme-linked secondary or detection antibodies can then be used to detect and assess the amount of CL-K1 in the sample tested. The intensity of the signal from the detectable label corresponds to the amount of enzyme present, and therefore the amount of CL-K1. Levels can be quantified, for example by densitometry.

The level of CL-K1 can also be measured by measuring the biological activity of CL-K1.

In some embodiments, the level of CL-K1 can also be measured by measuring the mRNA level of CL-K1. Methods of measuring mRNA levels include, but are not limited to, polymerase chain reaction (PCR, e.g., quantitative or semi-quantitative, or reverse transcription-PCR), hybridization assay, Northern blotting, primer extension, and ribonuclease protection.

The terms “sample”, “biological sample”, or “test sample” as used herein denote a sample taken or isolated from a biological organism, e.g., an animal or human. e.g., a blood sample or a plasma sample from a subject. Exemplary biological samples include, but are not limited to, a biofluid sample; a body fluid sample, blood (including whole blood); serum; plasma; urine; saliva; a biopsy and/or tissue sample etc. The term also includes a mixture of the above-mentioned samples. The term “sample” also includes untreated or pretreated (or pre-processed) biological samples. In some embodiments, a sample can comprise one or more cells from the subject. In some embodiments, the sample used for the assays, methods, and systems described herein can comprise a blood sample collected from a subject to be tested. In some embodiments, the sample used for the assays, methods, and systems described herein can comprise a plasma sample collected from a subject to be tested.

The test sample can be obtained by removing a sample from a subject, but can also be accomplished by using previously isolated samples (e.g. isolated at a prior time point and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample.

In some embodiments, the reference level can correspond to an average level of CL-K1 in a sample (e.g., plasma) of a normal healthy subject or a population of normal healthy subjects. This would be a “normal” level. For example, the average plasma CL-K1 level reported for healthy Japanese and Danish populations is about 340 ng/mL and about 284 ng/mL, respectively.

As used herein, the term “normal healthy subject” refers to a subject who has no symptoms of any diseases or disorders, or who is not identified with any diseases or disorders, or who is not on any medication treatment, or a subject who is identified as healthy by physicians based on medical examinations.

In some embodiments, the reference level can be at least one standard deviation (including, e.g., at least two standard deviations) above the average level of CL-K1 in a sample (e.g., plasma) of a normal healthy subject or a population of normal healthy subjects. In some embodiments, the reference level can be at least two standard deviations above the average level of CL-K1 in a sample (e.g., plasma) of a normal healthy subject or a population of normal healthy subjects. In these embodiments, any level above the reference level is considered to be significantly different from the average level of CL-K1 in a sample of a normal healthy subject or a population of normal healthy subjects.

In some embodiments, the reference level can be a level of CL-K1 in a control sample, a pooled sample of control individuals, or a numeric value or range of values based on the same. In some embodiments, the reference level (e.g., for a blood or plasma sample) is about 300 ng/mL or less, about 400 ng/mL or less, about 500 ng/mL or less, about 600 ng/mL or less, or about 700 ng/mL or less. In some embodiments, the reference level can be about 650 ng/mL or less. In some embodiments, the reference level can be about 619 ng/mL in plasma. It is also contemplated that a set of standards can be established with reference levels providing thresholds indicative of the severity of DIC or the risk level of developing DIC (e.g., highly likely, mildly likely, not likely, etc.).

In some embodiments, the reference level can be an average level of CL-K1 in a sample (e.g., plasma) of subject or a population of subjects without DIC. These subjects might have a disease other than DIC, a respiratory disease, or coagulopathy. For example, a subject without DIC can be diagnosed with a heart disease a gastrointestinal disease, a renal disease, diabetic milieu, an infection, or a neoplasm.

In some embodiments, the reference level can be at least one standard deviation (including, e.g., at least two standard deviations) above the average level of CL-K1 in a sample (e.g., plasma) of a subject or a population of subjects without DIC. In these embodiments, any level above the reference level is considered to be significantly different from the average level of CL-K1 in a sample of a subject or a population of subjects without DIC.

In some embodiments, the reference level can be a level of CL-K1 in a sample (e.g., blood) of the same subject measured at an earlier time point, e.g., before or during the treatment. In these embodiments, a physician monitors the subject's CL-K1 levels over time for, e.g., determining treatment efficacy or managing the risk of developing DIC.

In some embodiments, the level of CL-K1 measured in a sample from a subject identified as having DIC or a risk of developing DIC can be at least 5%, at least 10%, at least 20%, at least 30%, at least 40%, at least 50%, at least 60%, at least 70%, at least 80%, at least 90%, at least 100%, at least 150%, at least 200%, or at least 300% higher than the reference level.

It should be noted that the reference level can be different, depending on factors such as the sample type where the reference level is derived, gender, age, weight, and ethnicity. For example, in a subject, the CL-K1 level in a plasma sample can be different from that in a urine sample. Thus, reference levels accounting for these and other variables can provide added accuracy for the methods described herein.

In some embodiments, a subject determined to have a CL-K1 level in a test sample which is at least two standard deviations (S.D.) (e.g., 2 S.D., 3 S.D., or 4 S.D.) higher than a reference level can be at least 1.5 times more likely to develop DIC, as compared to the reference, e.g. at least 1.5 times, at least 2 times more likely, at least 3 times more likely, at least 4 more likely, or at least 5 times more likely. In these embodiments, the reference level can be the average level of CL-K1 in a population of normal healthy subjects.

In some embodiments, the assays and methods described herein can comprise determining an odds ratio for a subject based upon the CL-K1 level in a test sample obtained from that subject as compared to a reference level. The odds ratio can be calculated using methods known in the art and the odds ratio can be used to determine the relative risk of the subject developing DIC. In some embodiments, the odds ratio can be calculated by using a nominal logistic regression model and adjusted to age using a statistical analysis software (e.g., JMP software (SAS Institute)).

In some embodiments, when the subject is identified as having DIC, the method can further comprise providing to the subject a treatment appropriate for treating DIC. The treatment of DIC can include, e.g., controlling bleeding and clotting problems and optionally treating the underlying cause of DIC, if any. In some embodiments, treatments can vary with the underlying cause. Some examples of underlying causes include, but are not limited to, thrombosis, high risk of bleeding, and a primary hyperfibrinolytic state.

For example, in some embodiments, the underlying cause of DIC can be that thrombosis (e.g., arterial or venous thromboembolism) predominates in the subject with DIC. In these embodiments, the treatment for DIC can comprise administering to the subject an anticoagulant. An exemplary anticoagulant includes, but is not limited to, heparin.

In some embodiments, the treatment for DIC can comprise administering to the subject recombinant human activated protein C. For example, the subject can have both DIC and sepsis, and can be amenable to the treatment comprising recombinant human activated protein C. In general, subjects with high risk of bleeding should not be given recombinant human activated protein C.

In some embodiments, the treatment for DIC comprises administering to the subject recombinant factor VII. Trade names for recombinant factor VII include NovoSeven and AryoSeven.

In some embodiments, the treatment for DIC comprises administering to the subject a lysine analogue such as tranexamic acid. For example, a subject with the underlying cause of a primary hyperfibrinolytic state and present with severe bleeding can be amenable to the treatment comprising a lysine analogue.

In some embodiments, the treatment for DIC comprises transfusing platelets or plasma or plasma components in the subject. For example, the subject with DIC is present with bleeding or at high risk of bleeding and a platelet count of less than 5×10¹⁰/L.

It should be noted that the above treatment guideline is for illustration purpose only and is in no way limiting. Other treatment methods are disclosed, for example, in U.S. Pat. No. 8,652,477, US20050070480, WO2008044631, EP0519354, US20100152106, and U.S. Pat. No. 5,948,752, the contents of which are incorporated herein by reference.

In one aspect, the invention provides an assay comprising: measuring, in a sample obtained from a subject, a level of collectin kidney 1 (CL-K1); comparing the level of CL-K1 with a reference level; and identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.

In one aspect, the invention provides an assay comprising: contacting a plasma sample obtained from a subject with a detectable anti-CL-K1-antibody reagent; detecting the intensity of a detectable signal, wherein the intensity of the detectable signal indicates a level of CL-K1 in the subject; comparing the level of CL-K1 with a reference level; and identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level. In some embodiments, the antibody reagent is detectably labeled or capable of generating a detectable signal.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the antibody reagent. Alternatively, a detectable label can be linked such as by directly labeling a molecule that achieves binding to the antibody reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In one aspect, the invention provides a method of monitoring a risk of a subject of developing disseminated intravascular coagulation (DIC), the method comprising: measuring, at a first time point, a first level of collectin kidney 1 (CL-K1) in a first sample obtained from the subject; measuring, at a second time point, a second level of CL-K1 in a second sample obtained from the subject, wherein the second time point is later than the first time point, and wherein if the second level is significantly higher than the first level, then the risk of the subject developing DIC is identified as increased.

The CL-K1 levels (e.g., plasma CL-K1 levels) can be monitored, for example, once every day, once every few days (e.g., 2, 3, 4, 5, 6), once every week, once every two weeks, once every three weeks, once every month, or once every six months. In some embodiments, the CL-K1 level measured at an earlier time point is stored in a storage module in a computer system. The monitoring can be performed more frequently when there is an upward trend in the CL-K1 levels of the subject over time.

In one aspect, the invention provides a method of monitoring treatment progress in a subject suffering from disseminated intravascular coagulation (DIC), the method comprising: measuring, at a first time point, a first level of collectin kidney 1 (CL-K1) in a first sample obtained from the subject; measuring, at a second time point, a second level of CL-K1 in a second sample obtained from the subject, wherein the second time point is later than the first time point and after the administration of a therapeutic agent for DIC, and wherein if the second level is significantly lower than the first level, then the treatment is considered to be effective. As used herein, the term “therapeutic agent” refers to any therapeutically active substance that is administered to a subject to produce a desired, usually beneficial, effect. In some embodiments, the therapeutic agent is an anticoagulant. In some embodiments, the therapeutic agent is recombinant human activated protein C. In some embodiments, the therapeutic agent is recombinant factor VII. In some embodiments, the therapeutic agent is a lysine analogue. In some embodiments, the therapeutic agent is platelets, plasma, or plasma components.

In some embodiments, any of the assays or methods described herein can further comprise creating a report based on the CL-K1 level measured in the subject. Such reports can comprise the CL-K1 level in the subject, the normal level of CL-K1, whether the subject has DIC, or the risk of the subject developing DIC.

### Subjects

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein.

Preferably, the subject is a mammal. The mammal can be a human, non-human primate, mouse, rat, dog, cat, horse, or cow, but are not limited to these examples.

In some embodiments, a subject can be one who has been previously diagnosed with or identified as suffering from DIC. In some embodiments, a subject can be one who is at a risk of developing DIC and in need of monitoring. In some embodiments, a subject can be one who display one or more symptoms related to DIC. Symptoms related to DIC include, but are not limited to, bleeding, blood clots, bruising, and drop in blood pressure. In some embodiments, the subject can be one who has a family history of the family members suffering from DIC. In some embodiments, the subject can be one who is suspected of developing DIC. In some embodiments, the subject can be one who is undergoing treatment for DIC.

In some embodiments, the subject is a male human. In some embodiments, the subject is a female human.

In some embodiments, the subject has a vascular disease.

In some embodiments, the assays/methods of the present invention can be used to select a subject for treating DIC.

### Computer Systems

In some embodiments of the assays and/or methods described herein, the assay/method comprises or consists essentially of a system for determining (e.g. transforming and measuring) the CL-K1 level as described herein and comparing them to a reference level. If the comparison system, which can be a computer implemented system, indicates that the amount of the measured CL-K1 level is statistically higher than that of the reference amount, the subject from which the sample is collected can be identified as, e.g. having DIC or a risk of experiencing DIC.

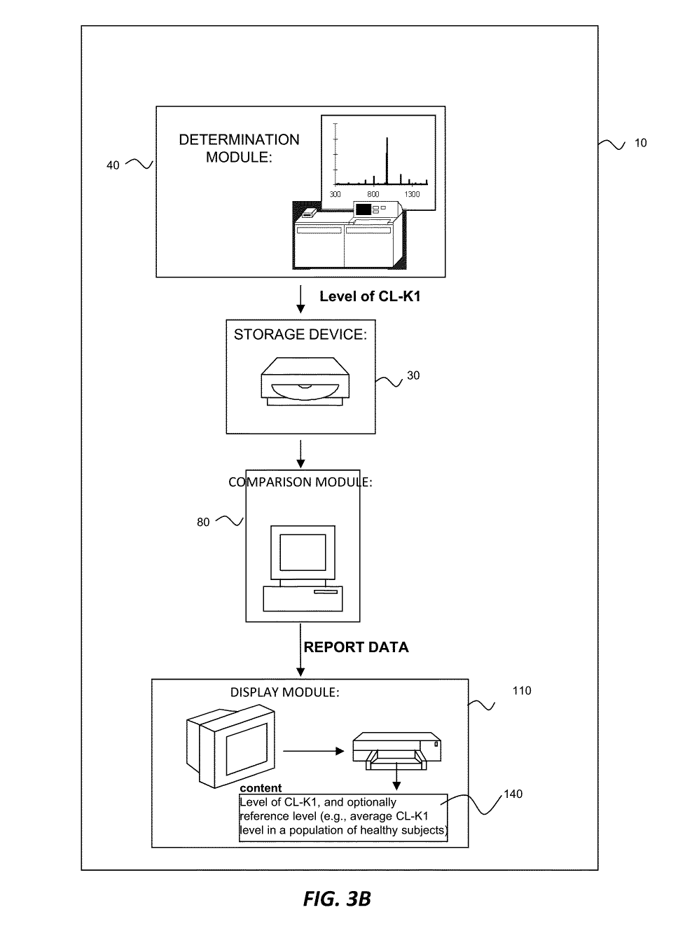

FIG. 3A depicts a device or a computer system 10 comprising one or more processors 20 and a memory 50 storing one or more programs 70 for execution by the one or more processors 20.

In some embodiments, the device or computer system 10 can further comprise a non-transitory computer-readable storage medium 200 storing the one or more programs 70 for execution by the one or more processors 20 of the device or computer system 10.

In some embodiments, the device or computer system 10 can further comprise one or more input devices 90, which can be configured to send or receive information to or from any one from the group consisting of: an external device (not shown), the one or more processors 20, the memory 50, the non-transitory computer-readable storage medium 200, and one or more output devices 130.

In some embodiments, the device or computer system 10 can further comprise one or more output devices 130, which can be configured to send or receive information to or from any one from the group consisting of: an external device (not shown), the one or more processors 20, the memory 50, and the non-transitory computer-readable storage medium 200.

In one embodiment, provided herein is a system comprising: (a) at least one memory containing at least one computer program adapted to control the operation of the computer system to implement a method that includes (i) a determination module configured measure the CL-K1 level in a test sample obtained from a subject; (ii) a storage module configured to store output data from the determination module; (iii) a computing module adapted to identify from the output data whether the measured CL-K1 level in the test sample obtained from the subject is higher, by a statistically significant amount, than a reference level, and to provide a retrieved content; (iv) a display module for displaying for retrieved content (e.g., the amount of the measured CL-K1 level, or whether the measured CL-K1 level is higher than the reference level); and (v) at least one processor for executing the computer program.

FIG. 3B depicts a device or a system (e.g., a computer system) for obtaining data from at least one sample obtained from one or more subjects. Accordingly, a system for analyzing a sample is provided herein. The system can be used to diagnose or prognose a condition or state of a condition in a subject. The system 10 comprises: (a) a determination module 40 configured to receive a sample and to determine the CL-K1 level in the sample; (b) a storage device 30 configured to store the CL-K1 level information from the determination module 40; (c) a comparison module 80 adapted to compare the sequence information stored on the storage device with a reference level, and to provide a comparison result, wherein the comparison result identifies whether the subject has DIC or a risk of developing DIC; and (d) a display module 110 for displaying a content based in part on the comparison result for the user, wherein the content is a signal indicative of a subject having DIC or a risk of developing DIC.

A tangible and non-transitory (e.g., no transitory forms of signal transmission) computer readable medium 200 having computer readable instructions recorded thereon to define software modules for implementing a method on a computer is also provided herein. In some embodiments, the software modules can include a comparison module and a display module for implementing a method on a computer. In some embodiments, the computer-readable medium 200 stores one or more programs for determining the CL-K1 level in a sample. In some embodiments, the computer-readable medium 200 stores one or more programs for determining a condition or state of a condition of a subject. The one or more programs for execution by one or more processors of a computer system comprises (a) instructions for comparing the measured CL-K1 level (from a sample) stored on a storage device with a reference level to provide a comparison result, wherein the comparison result identifies whether the subject has DIC or a risk of developing DIC; and (b) instructions for displaying a content based in part on the comparison result for the user, wherein the content is a signal indicative of a subject having DIC or a risk of developing DIC.

Embodiments can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing. Computer-readable storage medium do not include a signal.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the technology described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments can include at minimum a determination module, a storage module, a computing module, and a display module. The functional modules can be executed on one, or multiple, computers, or by using one, or multiple, computer networks. The determination module has computer executable instructions to provide e.g., levels of expression products etc in computer readable form.

The determination module can comprise any system for detecting a signal resulting from the detection of CL-K1 in a biological sample. In some embodiments, such systems can include an instrument, e.g., a plate reader for measuring absorbance. In some embodiments, such systems can include an instrument, e.g., the Cell Biosciences NANOPRO 1000™ System (Protein Simple; Santa Clara, Calif.) for quantitative measurement of proteins.

The information determined in the determination system can be read by the storage module. As used herein the “storage module” is intended to include any suitable computing or processing apparatus or other device configured or adapted for storing data or information. Examples of electronic apparatus suitable for use with the technology described herein include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, patient name, and numerical value of the CL-K1 level. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as levels of CL-K1 in healthy subjects. In some embodiments, the storage module stores the information such as levels of CL-K1 measured from the same subject in earlier time points.

The “computing module” can use a variety of available software programs and formats for computing the CL-K1 levels. Such algorithms are well established in the art. A skilled artisan is readily able to determine the appropriate algorithms based on the size and quality of the sample and type of data. The data analysis can be implemented in the computing module. In one embodiment, the computing module further comprises a comparison module, which compares the CL-K1 level in the test sample obtained from a subject as described herein with the reference level. By way of example, when the CL-K1 level in the test sample obtained from a subject is measured, a comparison module can compare or match the output data, e.g. with the reference level. In certain embodiments, the reference level has been pre-stored in the storage module. During the comparison or matching process, the comparison module can determine whether the CL-K1 level in the test sample obtained from a subject is higher than the reference level to a statistically significant degree. In various embodiments, the comparison module can be configured using existing commercially-available or freely-available software for comparison purpose, and may be optimized for particular data comparisons that are conducted.

The computing and/or comparison module, or any other module, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment, users can directly access data (via Hypertext links for example) residing on Internet databases using a HTML interface provided by Web browsers and Web servers.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, described herein is a computer system for determining the risk of a subject experiencing DIC, the system comprising: a determination module configured to measure a CL-K1 level in a test sample obtained from the subject; a storage module configured to store output data from the determination module; a comparison module adapted to compare the data stored on the storage module with a reference level, and to provide a retrieved content; and a display module for displaying the retrieved content. In some embodiments, the reference level can be the level of CL-K1 measured in the subject at an earlier time point.

In some embodiments, the content displayed on the display module can be the relative CL-K1 levels in the test sample obtained from a subject as compared to a reference level. In certain embodiments, the content displayed on the display module can indicate whether the CL-K1 level was found to be statistically significantly higher in the test sample obtained from a subject as compared to a reference level. In some embodiments, the content displayed on the display module can show the CL-K1 levels from the subject measured at multiple time points, e.g., in the form of a graph. In some embodiments, the content displayed on the display module can indicate whether the subject has DIC. In certain embodiments, the content displayed on the display module can indicate whether the subject has a risk of developing DIC. In certain embodiments, the content displayed on the display module can indicate whether the subject is in need of a treatment for DIC. In some embodiments, the content displayed on the display module can be a numerical value indicating one of these risks or probabilities. In such embodiments, the probability can be expressed in percentages or a fraction. For example, higher percentage or a fraction closer to 1 indicates a higher likelihood of a subject developing DIC. In some embodiments, the content displayed on the display module can be single word or phrases to qualitatively indicate a risk or probability. For example, a word “unlikely” can be used to indicate a lower risk for developing DIC, while “likely” can be used to indicate a high risk for developing DIC.

In one embodiment, the content based on the computing and/or comparison result is displayed on a computer monitor. In one embodiment, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the technology relating to determining the CL-K1 levels, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Devices/Kits

Provided herein are kits and devices for practicing the assays and methods described herein.

In some embodiments, described herein is a device for measuring the CL-K1 level in a test sample from a subject, the device comprising: (a) a CL-K1-specific antibody or antigen-binding portion thereof; and (b) at least one solid support, wherein the antibody or antigen-binding portion thereof of part (a) are deposited on the support. In some embodiments, the device can perform an assay in which an antibody-protein or antibody-peptide complex is formed. In some embodiments, the solid support can be in the format of a dipstick, a microfluidic chip, a multi-well plate or a cartridge. The kits or devices can employ immuno-based lateral flow technology to produce a signal.

In some embodiments, described herein is a kit comprising: a device as described in the preceding paragraph; and at least a detection antibody. In some embodiments, the detection antibody can be specific for CL-K1. In some embodiments, the detection antibody can be detectably labeled. In some embodiments, the kit can further comprise at least an agent for producing a detectable signal from the detection antibody.

In some embodiments, the kit or device can comprise a reference, e.g. a reference sample or reference signal. In some embodiments, the reference can comprise a plasma sample from a healthy subject.

Some embodiments of the invention are listed in the following paragraphs:

paragraph 1. An assay comprising:
(i) measuring, in a sample obtained from a subject, a level of collectin kidney 1 (CL-K1);
(ii) comparing the level of CL-K1 with a reference level; and
(iii) identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.
paragraph 2. The assay of paragraph 1, further comprising determining that the subject does not have a respiratory disease or coagulopathy other than DIC if the level of CL-K1 is above the reference level.
paragraph 3. The assay of paragraph 2, further comprising providing a treatment appropriate for treating DIC.
paragraph 4. The assay of paragraph 1, further comprising determining an underlying condition for DIC in the subject.
paragraph 5. The assay of paragraph 3, wherein the treatment comprises administering an anticoagulant.
paragraph 6. The assay of paragraph 3, wherein the treatment comprises administering recombinant factor VII.
paragraph 7. The assay of paragraph 3, wherein the treatment comprises transfusion of platelets or fresh frozen plasma.
paragraph 8. The assay of any of paragraphs 1-7, wherein the sample is a blood sample or a plasma sample.
paragraph 9. The assay of any of paragraphs 1-8, wherein the level of CL-K1 is measured by an immunoassay.
paragraph 10. The assay of paragraph 9, wherein the immunoassay is an enzyme-linked immunosorbent assay (ELISA).
paragraph 11. The assay of paragraph 10, wherein the ELISA is sandwich ELISA using two antibodies specific to CL-K1.
paragraph 12. The assay of paragraph 11, wherein the sandwich ELISA comprises:
(i) contacting the sample obtained from the subject with a first anti-CL-K1 antibody;
(ii) washing the sample to remove excess unbound antigen; and
(iii) contacting the sample with a second anti-CL-K1 antibody.
paragraph 13. The assay of any of paragraphs 1-12, wherein the reference level corresponds to an average CL-K1 level in a population of healthy subjects.
paragraph 14. The assay of any of paragraphs 1-12, wherein the reference level is two standard deviations above an average CL-K1 level in a population of healthy subjects.
paragraph 15. The assay of any of paragraphs 1-12, wherein the reference level corresponds to an average CL-K1 level in a population of subjects without DIC.
paragraph 16. The assay of paragraph 15, wherein the subject without DIC also does not have a respiratory disease or coagulopathy.
paragraph 17. The assay of any of paragraphs 1-12, wherein the reference level is two standard deviations above an average CL-K1 level in a population of subjects without DIC.
paragraph 18. The assay of any of paragraphs 1-12, wherein the reference level is about 619 ng/mL.
paragraph 19. The assay of any of paragraphs 1-12, wherein the reference level corresponds to a level of CL-K1 measured in the subject at an earlier time point.
paragraph 20. The assay of any of paragraphs 1-19, wherein the subject is a mammal.
paragraph 21. The assay of paragraph 20, wherein the mammal is a human.
paragraph 22. The assay of any of paragraphs 1-21, wherein the subject has a vascular disease.
paragraph 23. An assay comprising:
(i) contacting a plasma sample obtained from a subject with a detectable anti-CL-K1-antibody reagent;
(ii) detecting the intensity of a detectable signal, wherein the intensity of the detectable signal indicates a level of CL-K1 in the subject;
(iii) comparing the level of CL-K1 with a reference level; and
(iv) identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.
paragraph 24. The assay of paragraph 23, wherein the antibody reagent is detectably labeled or capable of generating a detectable signal.
paragraph 25. The assay of paragraph 24, further comprising administering, to the subject, a treatment selected from the group consisting of: an anticoagulant, recombinant factor VII, and transfusion of platelets or fresh frozen plasma.
paragraph 26. A method of identifying whether a subject has disseminated intravascular coagulation (DIC) or a risk of developing DIC, the method comprising:
(i) assaying, in a sample obtained from a subject, a level of collectin kidney 1 (CL-K1);
(ii) comparing the level of CL-K1 with a reference level; and
(iii) identifying the subject as (a) having disseminated intravascular coagulation (DIC) or a risk of developing DIC if the level of CL-K1 is above the reference level; and (b) not having DIC or a risk of developing DIC if the level of CL-K1 is at or below the reference level.
paragraph 27. The method of paragraph 26, further comprising determining that the subject does not have a respiratory disease or coagulopathy other than DIC if the level of CL-K1 is above the reference level.
paragraph 28. The method of paragraph 27, further comprising providing a treatment appropriate for treating DIC.
paragraph 29. The method of paragraph 26, further comprising determining an underlying condition for DIC in the subject.
paragraph 30. The method of paragraph 28, wherein the treatment comprises administering an anticoagulant.
paragraph 31. The method of paragraph 28, wherein the treatment comprises administering recombinant factor VII.
paragraph 32. The method of paragraph 28, wherein the treatment comprises transfusion of platelets or fresh frozen plasma.
paragraph 33. The method of any of paragraphs 26-32, wherein the sample is a blood sample or a plasma sample.
paragraph 34. The method of any of paragraphs 26-33, wherein the level of CL-K1 is measured by an immunoassay.
paragraph 35. The method of paragraph 34, wherein the immunoassay is an enzyme-linked immunosorbent method (ELISA).
paragraph 36. The method of paragraph 35, wherein the ELISA is sandwich ELISA using two antibodies specific to CL-K1.
paragraph 37. The method of paragraph 36, wherein the sandwich ELISA comprises:
(i) contacting the sample obtained from the subject with a first anti-CL-K1 antibody;
(ii) washing the sample to remove excess unbound antigen; and
(iii) contacting the sample with a second anti-CL-K1 antibody.
paragraph 38. The method of any of paragraphs 26-37, wherein the reference level corresponds to an average CL-K1 level in a population of healthy subjects.
paragraph 39. The method of any of paragraphs 26-37, wherein the reference level is two standard deviations above an average CL-K1 level in a population of healthy subjects.
paragraph 40. The method of any of paragraphs 26-37, wherein the reference level corresponds to an average CL-K1 level in a population of subjects without DIC.
paragraph 41. The method of paragraph 40, wherein the subject without DIC also does not have a respiratory disease or coagulopathy.
paragraph 42. The method of any of paragraphs 26-37, wherein the reference level is two standard deviations above the average CL-K1 level in a population of subjects without DIC.
paragraph 43. The method of any of paragraphs 26-37, wherein the reference level is about 619 ng/mL.
paragraph 44. The method of any of paragraphs 26-37, wherein the reference level corresponds to a level of CL-K1 measured in the subject at an earlier time point.
paragraph 45. The method of any of paragraphs 26-44, wherein the subject is a mammal.
paragraph 46. The method of paragraph 45, wherein the mammal is a human.
paragraph 47. The method of any of paragraphs 26-46, wherein the subject has a vascular disease.
paragraph 48. A method of monitoring treatment progress in a subject suffering from disseminated intravascular coagulation (DIC), the method comprising:
(i) measuring, at a first time point, a first level of collectin kidney 1 (CL-K1) in a first sample obtained from the subject;
(ii) measuring, at a second time point, a second level of CL-K1 in a second sample obtained from the subject, wherein the second time point is later than the first time point and after the administration of a therapeutic agent for DIC, and wherein if the second level is significantly lower than the first level, then the treatment is considered to be effective.
paragraph 49. The method of paragraph 48, wherein the first sample and the second sample are blood samples or plasma samples.
paragraph 50. The method of paragraph 48 or 49, wherein the subject is a human.
paragraph 51. A method of monitoring a risk of a subject of developing disseminated intravascular coagulation (DIC), the method comprising:
(i) measuring, at a first time point, a first level of collectin kidney 1 (CL-K1) in a first sample obtained from the subject;
(ii) measuring, at a second time point, a second level of CL-K1 in a second sample obtained from the subject, wherein the second time point is later than the first time point, and wherein if the second level is significantly higher than the first level, then the risk of the subject developing DIC is identified as increased.
paragraph 52. The method of paragraph 51, wherein the first sample and the second sample are blood samples or plasma samples.
paragraph 53. The method of paragraph 51 or 52, wherein the subject is a human.
paragraph 54. A computer system for determining whether a subject has disseminated intravascular coagulation (DIC) or a risk of developing DIC, the system comprising:
a determination module configured to measure a CL-K1 level in a test sample obtained from the subject;
a storage module configured to store output data from the determination module;
a comparison module adapted to compare the data stored on the storage module with a reference level, and to provide a retrieved content; and
a display module for displaying the retrieved content.
paragraph 55. The computer system of paragraph 54, wherein the determination module measures the intensity of a detectable signal from an immunoassay indicating the CL-K1 level.
paragraph 56. The computer system of paragraph 54 or 55, wherein the reference level corresponds to an average CL-K1 level in a population of healthy subjects.
paragraph 57. The computer system of paragraph 54 or 55, wherein the reference level is two standard deviations above an average CL-K1 level in a population of healthy subjects.
paragraph 58. The computer system of paragraph 54 or 55, wherein the reference level corresponds to an average CL-K1 level in a population of subjects without DIC.
paragraph 59. The computer system of paragraph 58, wherein the subject without DIC also does not have a respiratory disease or coagulopathy.
paragraph 60. The computer system of paragraph 54 or 55, wherein the reference level is two standard deviations above the average CL-K1 level in a population of subjects without DIC.
paragraph 61. The computer system of paragraph 54 or 55, wherein the reference level is about 619 ng/mL.
paragraph 62. The computer system of paragraph 54 or 55, wherein the reference level corresponds to a level of CL-K1 measured in the subject at an earlier time point.
paragraph 63. The computer system of any of paragraphs 54-62, wherein if the CL-K1 level is above the reference level, the subject is identified as having a risk of developing DIC.
paragraph 64. The computer system of any of paragraphs 54-62, wherein if the CL-K1 level is above the reference level, the subject is identified as having DIC and in need of treatment for DIC.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. DIC. The term “treating” includes reducing or alleviating at least one adverse effect or symptom of DIC. Treatment is generally “effective” if one or more symptoms or clinical markers are reduced. For example, treatment is considered effective if the level of CL-K1 is reduced to a level similar to that of a healthy person. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For example, a treatment is considered effective for a subject having DIC if the bleeding and/or clotting is reduced by at least 10%, at least 20%, at least 30%, at least 40%, at least 50%, at least 60%, at least 70%, at least 80%, at least 90%, or more, compared to the severity of bleeding and/or clotting before the treatment. In another example, a treatment is considered effective for a subject having DIC if the CL-K1 level is reduced by at least 10%, at least 20%, at least 30%, at least 40%, at least 50%, at least 60%, at least 70%, at least 80%, at least 90%, or more, compared to the CL-K1 level before the treatment.

As used herein, the term “administering,” refers to the placement of a compound into a subject by a method or route which results in at least partial delivery of the agent at a desired site. Pharmaceutical compositions can be administered by any appropriate route which results in an effective treatment in the subject.

As used herein, the term “statistically significant” or “significantly” refers to statistical significance and generally means a two standard deviation (2SD) or greater difference.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. In some embodiments, an antibody reagent “specific for” a particular target molecule can be an antibody reagent that specifically binds that target molecule.

The singular terms “a,” “an,” and “the” include plural referents unless context clearly indicates otherwise. Similarly, the word “or” is intended to include “and” unless the context clearly indicates otherwise.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); and Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with numeric values or percentages may mean±5% of the value being referred to. For example, about 100 means from 95 to 105.

It should be understood that this invention is not limited to the particular methodology, protocols, and reagents, etc., disclosed herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although any known methods, devices, and materials may be used in the practice or testing of the invention, the methods, devices, and materials in this regard are disclosed herein.

Although methods and materials similar or equivalent to those disclosed herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology disclosed herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The technology disclosed herein is further illustrated by the following examples which in no way should be construed as being further limiting.

### Example 1

### Elevated Plasma CL-K1 Level is Associated with a Risk of Developing Disseminated Intravascular Coagulation (DIC)

The body's first line of host defense is the innate immune system, which includes complement proteins, coagulation enzymes and pattern recognition molecules, [1], as well as effectors cells such as macrophages. CL-K1 (collectin kidney 1, also known as collectin 11) is a pattern recognition molecule of the complement system, and is a member of the collectin family, which also includes mannose binding lectin (MBL) and lung surfactant proteins [2,3]. These latter collectins were identified in the late 1980s and have been extensively investigated in both laboratory and clinical studies [4,5]. Much less is known about CL-K1, which was first identified in 2006, and reliable antibodies required for analysis have only recently become available [6,7]. Average plasma CL-K1 concentrations have been reported as 340 and 284 ng/ml among healthy Japanese and Danish populations, respectively, [6,7], which are approximately comparable. This is much lower than the reported MBL plasma concentration of 2 μg/mL of MBL [8,9], although the effects of this difference in plasma concentration may be diminished by differences in their synthesis, as MBL is produced primarily in the liver whereas CL-K1 is synthesized in a much wider distribution of tissues, including liver, lung, kidney, brain, and endothelial cells [2, 10, 11].

CL-K1, like other collectins, recognizes and binds to specific chemical components of microbes [2,12]. Based on these observations, it has been speculated that CL-K1 may share similar biologic functions with other collectins, in particular MBL. Earlier investigations have shown that MBL-associated serine protease (MASP)-1/3 in a complex with MBL activates coagulation through a thrombin-like serine protease activity [13-16]. Like MBL, CL-K1 has also been found in a complex with MASP-1/3, supporting the idea that CL-K1 also may be involved with coagulation [2,12].

Plasma CL-K1 concentration was measured in a total of 659 specimens, including 549 DIC patients, 82 non-DIC patients and 27 healthy volunteers. The mean plasma CL-K1 levels in these cohorts were 424, 238 and 265 ng/ml, respectively, with no significant difference in the latter two groups. The incidence of elevated plasma CL-K1 was significantly higher in the DIC patients compared to the non-DIC patients, resulting in an odds ratio of 1.929 (confidence interval 1.041-3.866). Infection, renal diseases, respiratory diseases, and cardiac diseases were more frequently observed in the DIC group than in the non-DIC group. In the DIC group, vascular diseases were associated with elevated plasma CL-K1 levels while age and acute illness had little effect on plasma CL-K1 levels. Independent of DIC, elevated plasma CL-K1 levels were associated with respiratory disease and coagulation disorders. These results suggest that specific diseases may affect CL-K1 synthesis in an organ dependent manner and that elevated plasma CL-K1 levels are associated with the presence of DIC. And thus elevated plasma CL-K1 level can be a new useful risk factor and a biomarker for the prediction of developing DIC.

### Materials and Methods

Plasma Samples and Patient Diagnoses.

Citrated plasma samples were collected and stored at −80° C.; a total of 659 specimens included 549 patients with DIC, 83 patients without DIC and 27 healthy volunteers. This study was retrospective, using clinical data retrieved from the hospital electronic medical records and surplus plasma samples from the clinical laboratories.

DIC was diagnosed by the presence of an abnormal biphasic waveform from activated partial thromboplastin time testing (aPTT), as described above [18]. The waveform is 98% specific for DIC (Toh 1998), unlike other laboratory tests which lack specificity for DIC. A biphasic waveform is characterized by a steeply negative initial slope, called slope_{—}1 (% T/sec). Abnormal slope_{—}1 is defined as less than −0.1% T/sec, with a normal slope_{—}1 defined as between −0.1 and +0.1, with reference values determined in the Coagulation Laboratory at the Massachusetts General Hospital. The aPTT waveform analysis was performed on an MDA-II coagulation analyzer (Tcoag/Diagnostica Stago, Parsippany N.J.). The MDA Coagulation Analyzer uses waveform analysis technology to automatically detect the presence of this abnormal waveform while running routine aPTT tests using Platelin L reagent [18].

Non-DIC patients were randomly selected hospital patients who did not have a clinical diagnosis of DIC, all of whom also had a normal aPTT waveform. In determining the DIC and non-DIC patients' medical conditions, respiratory diseases included acute respiratory distress syndrome (ARDS), other respiratory distress, respiratory failure, and pulmonary emboli. Vascular diseases included cerebrovascular disease and peripheral vascular diseases exclusive of coronary artery diseases, which were separately categorized as cardiac diseases. Coagulation disorders included any disorder of hemostasis including retroperitoneal, gastrointestinal, cerebrovascular or other hemorrhage, pulmonary emboli, deep vein thrombosis, heparin-induced thrombocytopenia, thrombosis, or stroke (ischemic, embolic or hemorrhagic). Gastrointestinal and renal diseases were also recorded, as well as diabetes or infection.

ELISA Assays.

Plasma CL-K1 was assayed using a previously established sandwich ELISA method using two CL-K1 specific antibodies, with minor modifications [7]. Briefly, 384 well plates were coated with 20 μL of an anti-CL-K1 rabbit polyclonal antibody. After rinsing and blocking, 20 μL of diluted plasma samples and standard were incubated in duplicate. All plasma samples used in this study were thawed only once. After rinsing, the wells were incubated with a biotinylated anti-CL-K1 monoclonal antibody followed by ABC-AP (Vector Lab, Burlingame, Calif.) and then developed using p-nitrophenyl phosphate substrate (Sigma-Aldrich). Reactions were assayed for absorbance at 405 nm using an M3 plate reader (Molecular Devices).

From assays performed using plasma specimens from 27 healthy volunteers, the median of plasma CL-K1 levels was 245 ng/mL with interquartile range 160-273 ng/mL. The highest concentration measured in this group was 772 ng/mL. The CL-K1 reference range was found to be <619 ng/ml (normal donor mean+2SD).

Plasma samples from a subpopulation of 216 patients (201 DIC and 15 non-DIC) were also assayed for plasma CRP, an acute phase protein, using ELISA (DuoSet, R&D Systems), according to the manufacturer's instructions.

Statistics.

Statistical analysis was performed using JMP software (SAS Institute, Cary, N.C.), and specific methods used are indicated in the table and figures. For a logistic regression analysis, plasma CL-K1 concentration was assigned to low and high, using a cut off of 619 ng/ml (mean+2SD of healthy normal volunteers [22,23]). As specified in the text below, p values less than 0.05 were considered to be statistically significant.

### Results

Analysis of Diseases Associated with the Presence of DIC.

The median age and gender ratios were similar between the DIC and the non-DIC patient groups (Table 1). These data do not reveal an association between age and gender and the presence of DIC. Compared to the non-DIC group, the DIC group patients had a significantly higher incidence of several diseases. Ranked from the highest incidence in our sample to the lowest, these included: infection, renal diseases, respiratory diseases, and cardiac diseases (Table 1).

TABLE 1
Patient demographics and diagnoses

| | | Group | | | | |
|---|---|---|---|---|---|---|
| | | Non-DIC (n = 83) | | DIC (n-549) | | P values^{b} |
| Age | Median (IQ25 - 75%)^{a} | 63.5 | (50.5-73.3) | 67.0 | (53.0-78.0) | 0.0886 |
| Gender | Female/Male (ratio) | 33/50 | (1/1.515) | 240/309 | (1/1.288) | 0.4878 |
| Diagnoses | Neoplasm | 18 | (21.7) | 123 | (22.4) | 1.000 |
| | Respiratory diseases | 11 | (13.3) | 164 | (29.9) | 0.0014 |
| | Cardiac diseases | 10 | (12.1) | 145 | (26.4) | 0.0038 |
| | Vascular diseases | 15 | (18.1) | 130 | (23.7) | 0.3265 |
| | Coagulation disorders | 9 | (10.8) | 102 | (18.6) | 0.0899 |
| | Gastrointestinal diseases | 12 | (14.5) | 96 | (17.5) | 0.6387 |
| | Renal diseases | 7 | (8.4) | 130 | (23.7) | 0.0009 |
| | Diabetic milieu | 12 | (14.5) | 91 | (16.6) | 0.7502 |
| | Infection | 17 | (37.0) | 322 | (63.7) | 0.0007 |

^{a}IQ, interquartile range
^{b}Fisher's Exact test, 2-tail

Plasma CL-K1 Levels Among the Non-DIC Patients are Similar to Normal Controls.

Plasma CL-K1 levels in the non-DIC group had a median of 176 ng/ml (Table 2), which were not statistically different from the healthy normal group which had a median of 245 ng/ml. The highest plasma CL-K1 levels sampled were also comparable, as these were measured at 772 and 869 ng/ml in the healthy normal and the non-DIC groups, respectively. These findings suggest that the non-DIC group was similar to normal controls with respect to plasma CL-K1 levels, and that the non-DIC patients could be used as a control group.

TABLE 2
Elevated plasma CL-K1 levels are associated with presence of DIC

| | | Median (IQ25-75%)^{a} | | High/Total^{b} | | P values^{c} | OR^{d} | 95% CI^{e} |
|---|---|---|---|---|---|---|---|---|
| Total | Non-DIC | 176 | (33-463) | 12/83 | (14.5) | | | |
| | DIC | 409 | (192-657) | 154/549 | (28.1) | 0.0074 | 1.929 | 1.041-3.866 |
| Female | Non-DIC | 164 | (47-517) | 6/33 | (18.2) | | | |
| | DIC | 417 | (169-666) | 71/240 | (29.6) | 0.2174 | 1.613 | 0.666-4.518 |
| Male | Non-DIC | 186 | (32-399) | 6/50 | (12.0) | | | |
| | DIC | 407 | (201-639) | 83/309 | (26.9) | 0.0223 | 2.228 | 0.960-6.091 |

^{a}Units are ng/mL, IQ indicates interquartile range.
^{b}“High” indicates number of patients with elevated plasma CL-K1 levels, defined as greater than 619 ng/ml; “Total” indicates total number of patients in the group. Numbers in parentheses indicate percent of those with elevated CL-K1 plasma levels.
^{c}Fisher's Exact Test.
^{d}Odds (OR) ratios were calculated by a nominal logistic regression model and adjusted to age.
^{e}CI, confidence interval.

Elevated Plasma CL-K1 Level is Associated with the Presence of DIC.

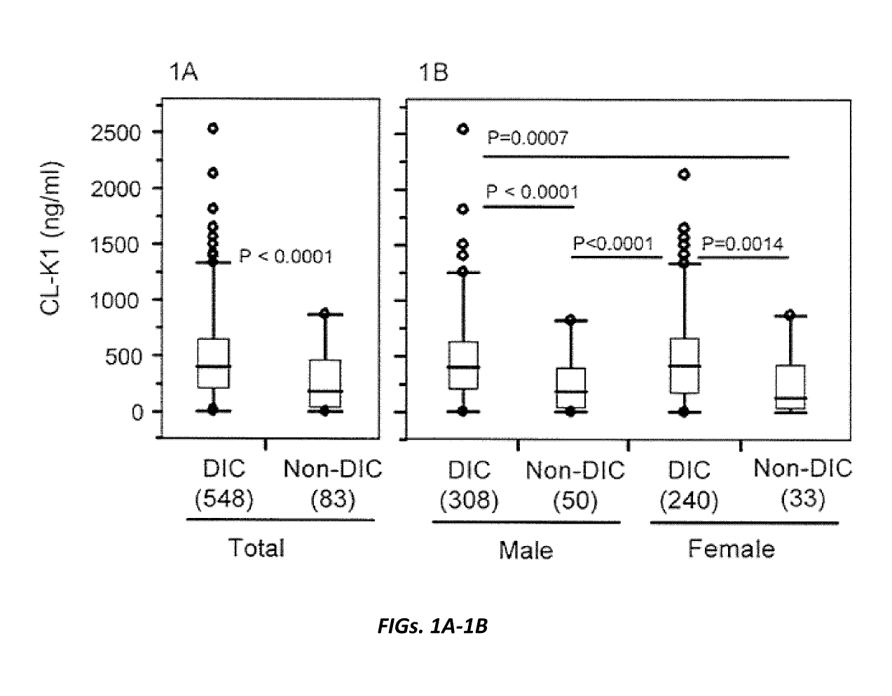

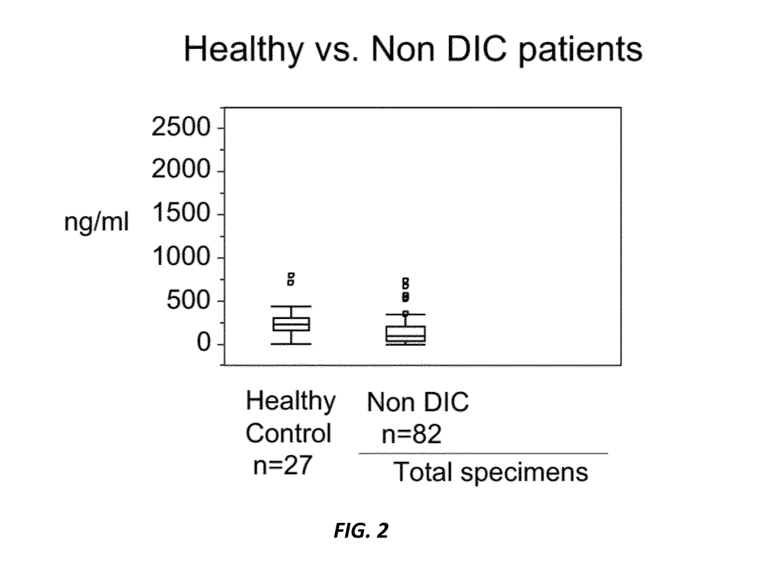

In contrast, plasma CL-K1 levels in the DIC group were significantly higher than the non-DIC group (FIG. 1A). A similar trend was observed when plasma CL-K1 levels were compared in different gender groups with statistical significance (FIG. 1B). In contrast, when plasma CL-K1 levels were compared for gender difference in the DIC or the non-DIC group (male DIC vs. female DIC or male non-DIC vs. female non-DIC), there was no statistical difference (p=0.655 and 0.856, respectively) (FIG. 1B). The median CL-K1 levels were more than 2 times higher in the DIC groups than the non-DIC groups, regardless of gender (Table 2).

Characteristics of the elevated plasma CL-K1 levels within the DIC and the non-DIC group were further analysed. A threshold for elevated plasma CL-K1 level was established as greater than 619 ng/ml, and logistic nominal regression analysis was used to evaluate the statistical significance of the high CL-K1 level. Plasma CL-K1 levels were significantly higher in the DIC group than the non-DIC group in all subjects, and there was a statistically significant excess of subjects with high CL-K1 levels (Table 2). A similar association was also observed in the male group, although this was not statistically significant in the female group (Table 2). Accordingly, the odds ratios (OR) were statistically significant for high CL-K1 plasma levels in all DIC subjects considered together and among male subjects, although the OR for high CL-K1 among DIC patients was not statistically significant among female subjects (Table 2).

In the DIC group, the effect of age on plasma CL-K1 levels was assessed by cohorts defined by grouping subjects into 20-year age intervals from 21 to 100 years old. Patients younger than 20 years of age were excluded due to small sample size, as there were only 17 subjects in that cohort and consequently statistical significance could not be achieved. As shown in Table 3, the median plasma CL-K1 concentrations were between 369 and 443 ng/ml and no statistical difference was obtained among different age cohorts. Likewise, the incidence of elevated plasma CL-K1 levels was between 21.5 and 34.3%, again with no statistically significant difference among the 4 age groups. These data demonstrate that in the DIC group, plasma CL-K1 levels and the incidence of elevated plasma CL-K1 concentrations were similar across age groups ranging from 21 to 100 years old.

TABLE 3
Plasma CL-K1 levels (ng/mL) by age cohort among patients with DIC

| Age | Median (IQ25-75%)^{a} | High/Total (%)^{b} |
|---|---|---|
| 21-40 | 404 (243-631) | 16/61 (26.3) |
| 41-60 | 443 (206-715) | 50/146 (34.3) |
| 61-80 | 412 (155-648) | 62/227 (27.3) |
| 81-100 | 369 (194-579) | 23/107 (21.5) |

^{a}IQ, interquartile range.
^{b}“High” indicates number of patients with elevated plasma CL-K1 levels, defined as greater than 619 ng/mL; “Total” indicates total number of patients in the group. Numbers in parentheses indicate percent of those with elevated CL-K1 plasma levels. There was no statistical difference among different age groups (assessed by Likelihood Ratio test).

Effect of Clinical Factors on Plasma CL-K1 Levels.

The association of other diseases and plasma CL-K1 levels were also analysed, in patients without or with DIC, by comparing the incidence of elevated plasma CL-K1 levels between the non-DIC and the DIC groups with respect to the most commonly identified co-existing diseases (Table 4). A statistically significant higher incidence of elevated plasma CL-K1 levels was observed only in patients with vascular diseases. Among all patients, both those without and with DIC, there was a statistically significant increased incidence of high plasma CL-K1 levels in patients with respiratory diseases and coagulopathies (Table 5).

TABLE 4
Incidence of elevated plasma CL-K I levels in
subjects with other diseases, without or with DIC

| | High/Total (%)^{a} | | |
|---|---|---|---|
| Diagnosis | Non-D1C | DIC | p values^{b} |
| Neoplasm | 5/18 (27.8) | 32/123 (26.1) | 1.000 |
| Respiratory diseases | 1/11 (10) | 54/164 (32.9) | 0.1765 |
| Cardiac diseases | 1/9 (10) | 39/145 (26.9) | 0.4547 |
| Vascular diseases | 0/15 (0) | 38/130 (29.2) | 0.0116 |
| Coagulation disorders | 2/9 (22.2) | 37/102 (36.3) | 0.4897 |
| Gastrointestinal diseases | 3/12 (25.0) | 33/96 (34.4) | 0.7472 |
| Renal diseases | 1/7 (14.3) | 41/130 (31.5) | 0.4380 |
| Diabetic milieu | 0/12 (0) | 23/91 (25.3) | 0.0636 |
| Infection | 4/17 (23.5) | 99/322 (30.8) | 0.6017 |

^{a}“High” indicates number of patients with elevated plasma CL-K1 levels, defined as greater than 619 ng/mL; “Total” indicates total number of patients in the group. Numbers in parentheses indicate percent of those with elevated CL-K1 plasma levels.
^{b}Wilcoxon/Kruskal-Wallis tests (Rank Sums).

TABLE 5
Incidence of elevated plasma CL-K I
levels by disease, independent of DIC

| | High/Total (%)^{a} | | |
|---|---|---|---|
| Diagnostics | Positive | Negative | P values^{b} |
| Neoplasm | 37/141 (23.2) | 129/491 (26.3) | 1.000 |
| Respiratory diseases | 551175 (31.4) | 111/457 (24.3) | 0.0070 |
| Cardiac diseases | 40/155 (25.8) | 126/477 (26.4) | 0.9167 |
| Vascular diseases | 38/145 (26.2) | 128/487 (26.3) | 1.000 |
| Coagulation disorders | 39/111 (35.1) | 127/521 (24.4) | 0.0238 |
| Gastrointestinal diseases | 36/108 (33.3) | 130/524 (24.8) | 0.0722 |
| Renal diseases | 42/137 (30.7) | 124/495 (25.1) | 0.1897 |
| Diabetic milieu | 23/103 (22.3) | 143/529 (27.0) | 0.3916 |
| Infection | 104/340 (30.6) | 49/213 (23.0) | 0.0633 |

^{a}“High” indicates number of patients with elevated plasma CL-K1 levels, defined as greater than 619 ng/mL; “Total” indicates total number of patients in the group. Numbers in parentheses indicate percent of those with elevated CL-K1 plasma levels.
^{b}Fisher's Exact Test, 2-tail.

The effects of acute illness were also analyzed for a subpopulation of patients. Acute illness was defined as plasma CRP levels more than 0.8 μg/mL (a reference value established by the clinical laboratory at Massachusetts General Hospital). The median CRP for the non-DIC and the DIC group was 0.7 (interquartile range 0-21.3) μg/mL and 36.5 (interquartile range 2.2-102) μg/mL, respectively. In patients with DIC, the incidence of elevated plasma CRP levels was 138 out of 201 (68.7%), which was significantly higher than the 5 out of 15 (33.3%) patients with elevated CRP in a group of patients without DIC (p=0.0090, Fisher's Exact Test). In comparison, the incidence of elevated plasma CL-K1 levels in patients with high versus normal CRP levels was 55/138 (40.0%) and 24/63 (28.1%), respectively, which is not statistically significant. These data suggest that although acute illness, defined by elevated plasma CRP levels, is associated with a higher incidence of DIC, acute illness is not associated with higher plasma CL-K1 levels.

### Discussion

CL-K1 was initially discovered in 2006, and there is a relative dearth of information regarding its biological function or clinical relevance [2]. The recent availability of anti-CL-K1 antibodies, which are required to perform ELISA and other immunochemical analyses [6,7], provide an important foundation for advancing both basic scientific and clinical investigation of CL-K1. In this current study, we measured the plasma CL-K1 concentration among healthy Americans, and found it to be 265±177 ng/ml, which is in the range of prior measurements of 340±130 ng/ml measured in healthy Japanese and 284 ng/ml measured in healthy Danes [6,7]. This result establishes that plasma CL-K1 concentrations in healthy Americans are similar to those Japanese and Danes. However, studies in other ethnic groups could demonstrate additional factors that may influence plasma CL-K1 levels [6,7].

The current study demonstrates that among non-DIC patients, CL-K1 levels are comparable to a healthy population of normal control subjects. In contrast, there is a higher average plasma CL-K1 level and a higher incidence of elevated plasma CL-K1 in a population of patients with DIC. In the DIC group, respiratory, cardiac, renal, and infectious diseases are frequently observed, however these diseases do not show association with elevated plasma CL-K1 levels. In regarding to renal diseases, our unpublished observations show that plasma CL-K1 levels among dialysis patients are not elevated, suggesting that renal disease alone do not increase plasma CL-K1 levels. Our results also reveal no statistically significant effect of age on plasma CL-K1 levels among patients with DIC, although age has been identified as an important associated factor in the development of other clinical complications [24]. Taken together, these results suggest that elevated plasma CL-K1 level is a biomarker for developing DIC.

DIC is a serious medical condition in which thrombosis (coagulation) and bleeding occur simultaneously. It has a strong association with multiple organ failure and very high mortality [17-19]. The results herein show that coagulation disorders are associated with elevated plasma CL-K1 levels independent of the presence of DIC, supporting the idea that CL-K1 may play a role in coagulation. In support of the hypothesis that collectins may play a role in both coagulation and immunity, other studies have demonstrated that MBL, also a collectin molecule, has a thrombin-like serine protease activity when it forms a complex with MASP-1/3 [13, 15, 16, 25]. This MBL-MASP-1/3 complex also activates the lectin complement pathway [14, 26, 27], demonstrating its role in the immune system. Intriguingly, a complex of CL-K1 and MASP-1/3 has been found in circulation that is capable of complement activation [2,12]. Taken together, these observations lead to the hypothesis that a complex of CL-K1 and MASP-1/3 may mediate coagulation [2,12]. The data herein reveal an association between elevated CL-K1 and the coagulopathic DIC condition, and further detailed studies are required in order to elucidate the mechanisms by which CL-K1, MASPs or their complexes activate and modulate coagulation.

CL-K1, like other collectins, recognizes and binds to specific chemical components found on microbes [2,12]. Microbial infection is strongly associated with coagulation disorders. Previously one manifestation of coagulopathy in DIC was demonstrated, the presence of a biphasic aPTT waveform, which provides an early and sensitive diagnostic test for DIC [17,18]. In this regard, the results presented here confirm these previous findings, as infection is strongly associated with DIC. Additionally, the results presented here demonstrate a trend toward increased plasma CL-K1 in infection, independent of DIC, although this did not meet a threshold for statistical significance. These observations taken together inform the larger hypothesis that the interactions among CL-K1, MASPs and microbes contribute to abnormal activation of coagulation.

To ensure that accurate results were obtained, aPTT waveforms were measured in a proper and timely manner and all plasma samples were collected contemporaneous with the inpatient admission. Measurement of CL-K1 was performed using ELISAs that are routinely performed in the art, similar to many of previously reported experiments [13, 28-31]. The results shown here are the first to show a relationship between plasma CL-K1 levels, DIC and other co-existing diseases. These associations warrant further clinical and laboratory investigations, and may yield important insights into the connections between immunity and coagulation.

It should be noted that in contrast to the average MBL plasma level of 2 μg/mL, CL-K1 has a significantly lower plasma concentration [7-9,32]. However, CL-K1 is synthesized by most organs, including the lung and vascular wall lining endothelial cells [2, 10, 11, 33], in contrast to MBL, which is produced primarily in the liver. Interestingly, a high incidence of elevated plasma CL-K1 levels is associated with respiratory diseases but not with vascular diseases. However, vascular diseases were commonly seen in association with DIC. The ubiquitous presence of CL-K1 throughout the body may reflect contribute to its ability to participate in a systemic response, even though circulating levels of CL-K1 are relatively low. Consequently, and consistent with the observations presented here, CL-K1 may play a key role throughout the host defense and coagulation systems.

It is not known whether CL-K1 is an acute phase protein or how its synthesis is modulated, reflecting the relative paucity of published data. Given the similar levels of CL-K1 in healthy control subjects and hospital patients without DIC, it is possible that CL-K1 is not an acute phase protein, although elevations of CL-K1 in DIC suggest that there may be a regulated increase in its production. Prospective studies with longitudinal measurements in well-defined patient cohorts would be useful to further establish these findings. In contrast to the recent report on the genetic deficiency of CL-K1, our results suggest that regulatory gene sequences most likely exist and contribute to increased production and/or secretion of the CL-K1 protein [11,36]. Further investigations are required to understand the molecular mechanisms involved in this regulation, such as the specific stimuli, mediators and effector cells of increased CL-K1 production.

In conclusion, based on the studies and data provided herein, elevated plasma CL-K1 level can be a useful new biomarker for the prediction of developing DIC. CL-K1, as a member of the collectin family of the innate immune system, may play an important role in host defense and the maintenance of homeostasis in health and disease.

### REFERENCES

1. Thiel S, Takahashi K (2013) Collectins. In: Encyclopedia of Life Sciences. John Wiley & Sons, Ltd, Chichester,
2. Keshi H, Sakamoto T, Kawai T, Ohtani K, Katoh T, Jang S J, Motomura W, Yoshizaki T, Fukuda M, Koyama S, Fukuzawa J, Fukuoh A, Yoshida I, Suzuki Y, Wakamiya N (2006) Identification and characterization of a novel human collectin CL-K1. Microbiol Immunol 50 (12):1001-1013. doi:JST.JSTAGE/mandi/50.1001 [pii]
3. Ohtani K, Suzuki Y, Wakamiya N (2012) Biological functions of the novel collectins CL-L1, CL-K1, and CL-P1. J Biomed Biotechnol 2012:493945.
4. Thiel S (2007) Complement activating soluble pattern recognition molecules with collagen-like regions, mannan-binding lectin, ficolins and associated proteins. Molecular Immunology 44 (16):3875-3888
5. Takahashi K (2008) Lessons learned from murine models of mannose-binding lectin deficiency. Biochemical Society Transactions 36 (Pt 6):1487-1490.
6. Selman L, Henriksen M L, Brandt J, Palarasah Y, Waters A, Beales P L, Holmskov U, Jorgensen T J, Nielsen C, Skjodt K, Hansen S (2012) An enzyme-linked immunosorbent assay (ELISA) for quantification of human collectin 11 (CL-11, CL-K1). J Immunol Methods 375 (1-2):182-188.
7. Yoshizaki T, Ohtani K, Motomura W, Jang S J, Mori K, Kitamoto N, Yoshida I, Suzuki Y, Wakamiya N (2012) Comparison of human blood concentrations of collectin kidney 1 and mannan-binding lectin. J Biochem 151 (1):57-64.
8. Steffensen R, Thiel S, Varming K, Jersild C, Jensenius J C (2000) Detection of structural gene mutations and promoter polymorphisms in the mannan-binding lectin (MBL) gene by polymerase chain reaction with sequence-specific primers. J Immunol Methods 241 (1-2):33-42
9. Garred P, Larsen F, Madsen H O, Koch C (2003) Mannose-binding lectin deficiency—revisited. Mol Immunol 40 (2-4):73-84
10. Uemura K, Saka M, Nakagawa T, Kawasaki N, Thiel S, Jensenius J C, Kawasaki T (2002) L-MBP is expressed in epithelial cells of mouse small intestine. J Immunol 169 (12):6945-6950
11. Rooryck C, Diaz-Font A, Osborn D P, Chabchoub E, Hernandez-Hernandez V, Shamseldin H, Kenny J, Waters A, Jenkins D, Kaissi A A, Leal G F, Dallapiccola B, Carnevale F, Bitner-Glindzicz M, Lees M, Hennekam R, Stanier P, Burns A J, Peeters H, Alkuraya F S, Beales P L (2011) Mutations in lectin complement pathway genes COLEC11 and MASP1 cause 3MC syndrome. Nat Genet 43 (3):197-203.
12. Hansen S, Selman L, Palaniyar N, Ziegler K, Brandt J, Kliem A, Jonasson M, Skjoedt M O, Nielsen O, Hartshorn K, Jorgensen T J, Skjodt K, Holmskov U (2010) Collectin 11 (CL-11, CL-K1) is a MASP-1/3-associated plasma collectin with microbial-binding activity. J Immunol 185 (10):6096-6104.
13. Takahashi K, Chang W C, Takahashi M, Pavlov V, Ishida Y, La Bonte L, Shi L, Fujita T, Stahl G L, Van Cott E M (2011) Mannose-binding lectin and its associated proteases (MASPs) mediate coagulation and its deficiency is a risk factor in developing complications from infection, including disseminated intravascular coagulation. Immunobiology 216 (1-2):96-102.
14. Sekine H, Takahashi M, Iwaki D, Fujita T (2013) The role of MASP-1/3 in complement activation. Adv Exp Med Biol 735:41-53
15. Gulla K C, Gupta K, Krarup A, Gal P, Schwaeble W J, Sim R B, O'Connor C D, Hajela K (2010) Activation of mannan-binding lectin-associated serine proteases leads to generation of a fibrin clot. Immunology 129 (4):482-495.
16. Presanis J S, Hajela K, Ambrus G, Gal P, Sim R B (2004) Differential substrate and inhibitor profiles for human MASP-1 and MASP-2. Mol Immunol 40 (13):921-929
17. Toh C H, Ticknor L O, Downey C, Giles A R, Paton R C, Wenstone R (2003) Early identification of sepsis and mortality risks through simple, rapid clot-waveform analysis. Implications of lipoprotein-complexed C reactive protein formation. Intensive Care Med 29 (1):55-61.
18. Smith E Y, Charles L A, Van Cott E M (2004) Biphasic activated partial thromboplastin time waveform and adverse events in non-intensive care unit patients. Am J Clin Pathol 121 (1):138-141.
19. Penner J A (1998) Disseminated intravascular coagulation in patients with multiple organ failure of non-septic origin. Semin Thromb Hemost 24 (1):45-52.
20. Matsumoto T, Wada H, Nishioka Y, Nishio M, Abe Y, Nishioka J, Kamikura Y, Sase T, Kaneko T, Houdijk W P, Nobori T, Shiku H (2006) Frequency of abnormal biphasic aPTT clot waveforms in patients with underlying disorders associated with disseminated intravascular coagulation. Clin Appl Thromb Hemost 12 (2):185-192
21. Toh C H, Samis J, Downey C, Walker J, Becker L, Brufatto N, Tejidor L, Jones G, Houdijk W, Giles A, Koschinsky M, Ticknor L O, Paton R, Wenstone R, Nesheim M (2002) Biphasic transmittance waveform in the APTT coagulation assay is due to the formation of a Ca(++)-dependent complex of C-reactive protein with very-low-density lipoprotein and is a novel marker of impending disseminated intravascular coagulation. Blood 100 (7):2522-2529.
22. Krause R D, Anand V D, Gruemer H D, Willke T A (1975) The impact of laboratory error on the normal range: a Bayesian model. Clin Chem 21 (3):321-324
23. Hapke M, Patil K (1981) The establishment of normal limits for serum proteins measured by the rate nephelometer. Concepts of normality revisited. Hum Pathol 12 (11):1011-1015
24. Stemerman M B (1985) Coagulation in the elderly. Clin Geriatr Med 1 (4):869-885
25. La Bonte L R, Pavlov V I, Tan Y S, Takahashi K, Takahashi M, Banda N K, Zou C, Fujita T, Stahl G L (2012) Mannose-binding lectin-associated serine protease-1 is a significant contributor to coagulation in a murine model of occlusive thrombosis. J Immunol 188 (2):885-891.
26. Thiel S, Vorup-Jensen T, Stover C M, Schwaeble W, Laursen S B, Poulsen K, Willis A C, Eggleton P, Hansen S, Holmskov U, Reid K B, Jensenius J C (1997) A second serine protease associated with mannan-binding lectin that activates complement. Nature 386 (6624):506-510
27. Matsushita M (2009) Ficolins: complement-activating lectins involved in innate immunity. J Innate Immun 2 (1):24-32.
28. Takahashi K, Ezekowitz R A (2005) The role of the mannose-binding lectin in innate immunity. Clin Infect Dis 41 Suppl 7:S440-444
29. Moller-Kristensen M, Hamblin M R, Thiel S, Jensenius J C, Takahashi K (2007) Burn injury reveals altered phenotype in mannan-binding lectin-deficient mice. J Invest Dermatol 127 (6):1524-1531
30. Chang W C, White M R, Moyo P, McClear S, Thiel S, Hartshorn K L, Takahashi K (2010) Lack of the pattern recognition molecule mannose-binding lectin increases susceptibility to influenza A virus infection. BMC Immunol 11 (1):64.
31. Larvie M, Shoup T, Chang W C, Chigweshe L, Hartshorn K, White M R, Stahl G L, Elmaleh D R, Takahashi K (2012) Mannose-binding lectin binds to amyloid beta protein and modulates inflammation. J Biomed Biotechnol 2012:929803.
32. Ip W K, Lau Y L, Chan S Y, Mok C C, Chan D, Tong K K, Lau C S (2000) Mannose-binding lectin and rheumatoid arthritis in southern Chinese. Arthritis Rheum 43 (8):1679-1687
33. Motomura W, Yoshizaki T, Ohtani K, Okumura T, Fukuda M, Fukuzawa J, Mori K, Jang S J, Nomura N, Yoshida I, Suzuki Y, Kohgo Y, Wakamiya N (2008) Immunolocalization of a novel collectin CL-K1 in murine tissues. J Histochem Cytochem 56 (3):243-252.
34. Knoebl P (2010) Blood coagulation disorders in septic patients. Wien Med Wochenschr 160 (5-6):129-138.
35. Levi M, de Jonge E, van der Poll T (2003) Sepsis and disseminated intravascular coagulation. J Thromb Thrombolysis 16 (1-2):43-47.
36. Shi L, Takahashi K, Dundee J, Shahroor-Karni S, Thiel S, Jensenius J C, Gad F, Hamblin M R, Sastry K N, Ezekowitz R A (2004) Mannose-binding lectin-deficient mice are susceptible to infection with Staphylococcus aureus. J Exp Med 199 (10):1379-1390

What is claimed is:

1. An assay comprising:
(i) measuring, in a sample obtained from a subject, a level of collectin kidney 1 (CL-K1);
(ii) comparing the level of CL-K1 with a reference level; and
(iii) identifying the subject as (a) having disseminated intravascular coagulation (DIC) or a risk of developing DIC if the level of CL-K1 is above the reference level;
and (b) not having DIC or a risk of developing DIC if the level of CL-K1 is at or below the reference level.

2. The assay of claim 1, further comprising determining that the subject does not have a respiratory disease or coagulopathy other than DIC if the level of CL-K1 is above the reference level.

3. The assay of claim 2, further comprising providing a treatment appropriate for treating DIC.

4. The assay of claim 1, further comprising determining an underlying condition for DIC in the subject.

5. The assay of claim 3, wherein the treatment comprises administering an anticoagulant.

6. The assay of claim 3, wherein the treatment comprises administering recombinant factor VII.

7. The assay of claim 3, wherein the treatment comprises transfusion of platelets or fresh frozen plasma.

8. The assay of claim 1, wherein the sample is a blood sample or a plasma sample.

9. The assay of claim 1, wherein the level of CL-K1 is measured by an immunoassay.

10. The assay of claim 9, wherein the immunoassay is an enzyme-linked immunosorbent assay (ELISA).

11. The assay of claim 10, wherein the ELISA is sandwich ELISA using two antibodies specific to CL-K1.

12. The assay of claim 1, wherein the reference level corresponds to an average CL-K1 level in a population of healthy subjects.

13. The assay of claim 1, wherein the reference level is two standard deviations above an average CL-K1 level in a population of healthy subjects.

14. The assay of any of claim 1, wherein the reference level corresponds to an average CL-K1 level in a population of subjects without DIC.

15. The assay of claim 14, wherein the subject without DIC also does not have a respiratory disease or coagulopathy.

16. The assay of claim 1, wherein the reference level is two standard deviations above an average CL-K1 level in a population of subjects without DIC.

17. The assay of claim 1, wherein the reference level is about 619 ng/mL.

18. The assay of claim 1, wherein the subject is a human.

19. A method of monitoring treatment progress in a subject suffering from disseminated intravascular coagulation (DIC), the method comprising:
(i) measuring, at a first time point, a first level of collectin kidney 1 (CL-K1) in a first sample obtained from the subject;
(ii) measuring, at a second time point, a second level of CL-K1 in a second sample obtained from the subject, wherein the second time point is later than the first time point and after the administration of a therapeutic agent for DIC, and wherein if the second level is significantly lower than the first level, then the treatment is considered to be effective.

20. A method of monitoring a risk of a subject of developing disseminated intravascular coagulation (DIC), the method comprising:
(i) measuring, at a first time point, a first level of collectin kidney 1 (CL-K1) in a first sample obtained from the subject;
(ii) measuring, at a second time point, a second level of CL-K1 in a second sample obtained from the subject, wherein the second time point is later than the first time point, and wherein if the second level is significantly higher than the first level, then the risk of the subject developing DIC is identified as increased.
